(19) United States
(12) Patent Application Publication
(10) Pub. No.: US 2004/0152988 A1
(43) Pub. Date: Aug. 5, 2004

(54) Capsule imaging system

(76) Inventor: John Paul Weirich, Palo Alto, CA (US)

Correspondence Address: JOHN WEIRICH, 524 KENDALL #3, PALO ALTO, CA, 94306, US

(21) Appl. No.: 10/729,725

(22) Filed: Dec. 4, 2003

Related U.S. Application Data
(60) Provisional application No. 60/443,620, filed on Jan. 31, 2003.

(51) Int. Cl. A61B 6/00

(52) U.S. Cl. 600/473; 128/903; 600/476

(57) ABSTRACT

The capsule imaging system provides medical personnel with a device to examine the gastro-intestinal system and nearby tissues, using ultra-wideband imaging technology. The subject swallows the ultra-wideband sensor equipped capsule, then the imaging components visualize the GI lumen and nearby structures as the capsule travels through the GI tract. Additionally, the system provides ultraviolet and infrared imaging. Medical personnel and subject benefit from the new imaging perspectives provided by the present invention.

### CROSS-REFERENCE TO RELATED APPLICATIONS

This application is related to and claims priority from U.S. Provisional Patent Application No. 60/443,620, titled “Ultra-wideband Radar Gastro-Intestinal Imaging System”, filed on Jan. 31, 2003, which is hereby incorporated herein by reference for all purposes.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND OF THE INVENTION

The invention relates generally to in vivo imaging devices for the digestive system and more particularly to in vivo capsule imaging devices utilizing ultra-wideband (UWB) radar sensors.

A familiar instrument for imaging the digestive system is the endoscope which is a long flexible maneuverable tube that is inserted into either end of the gastrointestinal (GI) tract of the subject. The tip of an endoscope is equipped with a video camera or optical fibers which enable viewing of the interior of the digestive tract. A limitation of the endoscope is that it is not flexible enough to allow its passage throughout the entire GI tract, consequently only reachable areas are viewable.

More recently, capsule-shaped devices holding a CCD video camera, transmitter, and power supply, like the one described in U.S. Pat. No. 5,604,531, have been used to image the GI tract. A device like this takes visible-light pictures of the interior wall of the GI tract then transmits picture signals to a receiving device worn by the subject.

One limitation of such a capsule imaging device is that it only provides viewing of the top surface of the interior lumen of the GI tract that is illuminated visible-light. Folded or creased areas where visible-light cannot reach cannot be imaged. Another limitation is that visible-light viewing cannot always detect telling features necessary to recognize diseased tissues.

Visible-light cannot penetrate tissue significantly, so often x-ray imaging is employed to provide details about the interior features of body parts, for example, x-ray images of teeth and mammography.

Another category of imaging devices that use frequencies different from x-rays, is ultra wideband (UWB) devices. These devices use UWB radar sensor circuits that operate in the 3.1-10.6 Ghz range and function generally as described in U.S. Pat. Nos. 5,757,320, 5,805,110, or 5,774,091. One example of an UWB medical imaging device is described in U.S. Pat. No. 5,668,555, “Imaging System and Apparatus”, by Jon E. Starr, filed Sep. 1, 1995.

The size of an UWB radar sensor can be reduced enough so it can be encapsulated in a small swallowable capsule structure, by forming most of the electrical circuitry on an integrated circuit chip. This miniaturization of circuitry has been demonstrated by Time Domain Corporation with its PulsOn chipset, and the Aether4 receiver and Driver2 transmitter chips by Aether Wire and Location Inc.

There are various problems associated with different imaging technologies to examine the GI tract. Some of these problems include: MRI and CAT scanning equipment is bulky and expensive; X-ray imaging is expensive and causes radiation harm to body tissues; Endoscopy cannot reach the small intestine and can cause tissue perforations or abrasions by its mechanical movements; and visual-light capsule imaging cannot reveal tissue features beyond the interior surface of the GI tract tube.

Additionally each of these technologies can only examine tissues that react to the particular electromagnetic (EM) frequencies it uses in imaging, thus less reactive diseased tissues will not be discovered in using them. Therefore it is necessary to have additional diagnostic imaging tools available that can more readily reveal diseased tissues, such tools as the present invention which does imaging in the 3.1-10.6 Ghz UWB, ultraviolet, and infrared frequency ranges.

### BRIEF SUMMARY OF THE INVENTION

It is an object of the present invention to provide an inexpensive capsule-shaped ultra-wideband (UWB) radar sensor system that enables imaging of the gastro-intestinal (GI) digestive tract and body tissues close by the GI tract, as the capsule passes through the entire length of the tract, after being swallowed by the subject.

Another object is to provide ultraviolet (UV) and infrared (IR) imaging of the GI tract and tissues close to the GI tract, by using UV or IR frequency electromagnetic (EM) waves as the imaging wave for the radar sensor.

In accordance with a preferred embodiment of the present invention, there is provided a capsule shaped UWB imaging device that is small enough to be swallowed by the subject. The capsule is shaped such that it passes through the GI tract in a natural way, like food does. This capsule imaging device includes UWB radar sensor circuitry, controlling circuitry, a radio transceiver, and a power source.

Additionally, the preferred embodiment of the present invention includes a wearable signal reception and storage device taking the general form of a vest-type garment, and further includes a computer system which processes the stored imaging information into a readily understandable format.

In accordance with an alternative embodiment of the present invention, the emitting antenna of the UWB radar sensor circuitry is replaced with an UV frequency light emitting diode (LED), and an UV frequency photodiode detector is substituted for the receiving antenna of the UWB sensor circuitry. This alternative embodiment uses UV frequency waves for imaging, as opposed to the 3.1-10.6 Ghz UWB waves used in the preferred embodiment. The UWB circuitry drives the UV LED to emit the imaging waves, and relatedly the UWB circuitry drives the timing of the UV photodiode detector as to when to receive reflected UV waves.

Additionally, in the alternative embodiment, the capsule shell is sufficiently transparent to UV waves, to allow these waves to pass through the shell such that waves reflected from the tissues back to the capsule, can be detected by the UV photodiode inside it.

Another alternative embodiment of the present invention, uses an infrared (IR) LED as the imaging wave emitter, and uses an infrared photodiode detector as the imaging wave receptor. Thereby, this alternative embodiment uses IR frequency waves for imaging purposes.

The preferred embodiment and alternative embodiments of the present invention include a vest-type receiving system that is worn like a vest by the subject. This receiving system includes antennae to gather the signals of imaging data transmitted from the capsule device, a power supply battery, controlling circuitry, and a data storage unit.

The capsule device transmits radar sensor imaging information signals through the body of the subject as it travels down the GI tract. These imaging information signals are received by the antennae embedded in the vest-like garment worn by the subject. These received signal data are then saved in the data storage device by means of controlling circuitry. The data storage device could be a small disk drive or a recording tape machine, for example. Thus the radar sensor imaging information transmitted from the capsule is saved for processing by the computer imaging system component of the present invention.

Another alternative is that the receiving antennae, controlling circuitry, storqge device, and power supply are held by supports not worn by the subject, yet they still are in operable contact with the transmitting capsule imaging device within the subject.

Moreover, both the preferred and alternative embodiments of the present invention, include a computer system that is programmed to process the information saved by the vest-like receiving-storage system just described above. The information saved by the data storage component of the vest receiving system is input into the computer system. The software programming of the computer system processes this imaging data into understandable output reports like graphs, tables, pictures, video streams, and other useful forms. These outputs could be shown on the computer display, or printed on paper, or saved on disk, for example.

Use of the present invention provides physicians and others with an additional imaging tool that can help them determine the condition of the subject's GI system. The present invention avoids the drawbacks of other imaging systems, disadvantages such as excessive size and fixed location, mechanical injury, radiation damage, high cost, and tissue inactivity with imaging wave frequency, among others.

### BRIEF DESCRIPTION OF THE SEVERAL VIEWS OF THE DRAWING

FIG. 1 is a side view of the capsule shaped shell that holds the UWB radar sensor system.

FIG. 2 is a schematic diagram of the components of the UWB radar sensor system within the capsule shell.

FIG. 3 is a schematic diagram of the UV radar sensor system of an alternative embodiment of the present invention within the capsule shell.

FIG. 4 is an illustration of the vest-style receiving system.

FIG. 5 is an illustration of the computer imaging processing system.

FIG. 6 is a cut-away view of the capsule with connecting electrodes.

### DETAILED DESCRIPTION OF THE INVENTION

Recent developments in construction of ultra-wideband (UWB) devices similar to those described in U.S. Pat. Nos. 5,274,271 and 5,361,070 have enabled designers to shrink their size to microchip dimensions. As examples, the commercial firms Time Domain Corp., XtremeSpectrum Inc., and Aether Wire and Location Inc. have all created microchip sized UWB transmitting and receiving integrated circuit chips.

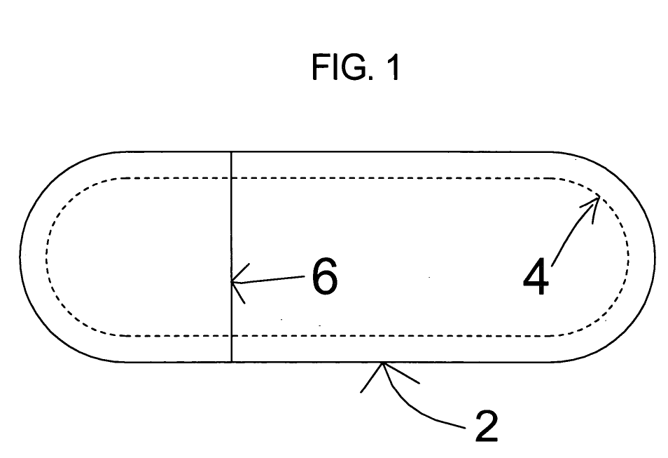

Referring now to FIG. 1, with this reduction in size, it is now possible to fit an entire UWB radar sensor system into a small swallowable capsule 2. The capsule 2 is hollow as the inside wall of the shell is indicated at 4. The components of the UWB radar sensor system are fitted into the hollow shell, then the shell is capped and the cap is glued on 6 to secure the contents within the non-digestible capsule while it travels through the gastro-intestinal (GI) digestive system. The shell could be made of a non-digestible polymer and the cap part has a watertight seal with the rest of the capsule. The capsule is shaped so it passes through the GI tract of the subject just as naturally as food does.

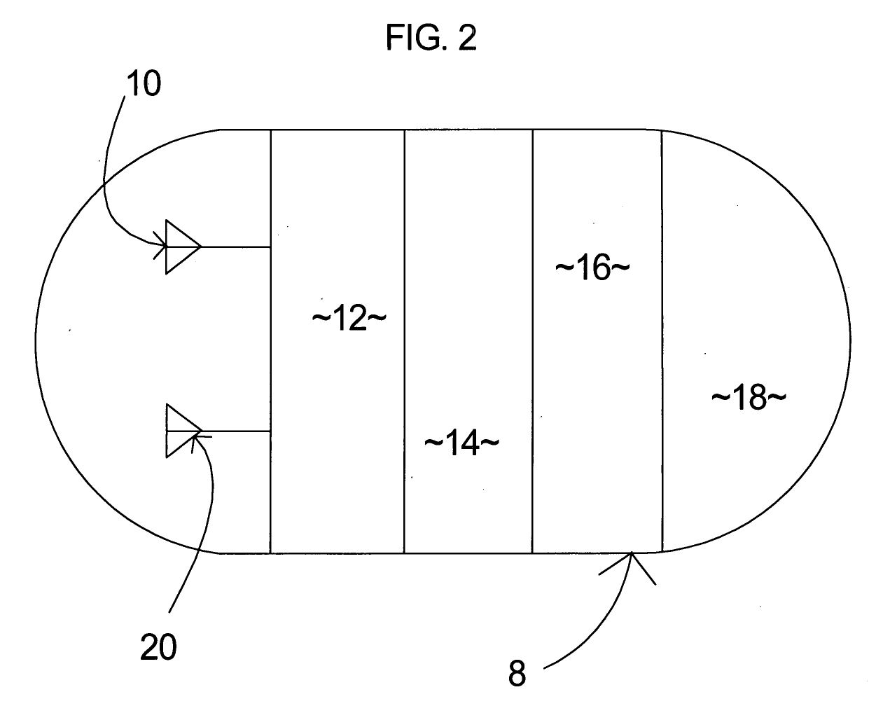

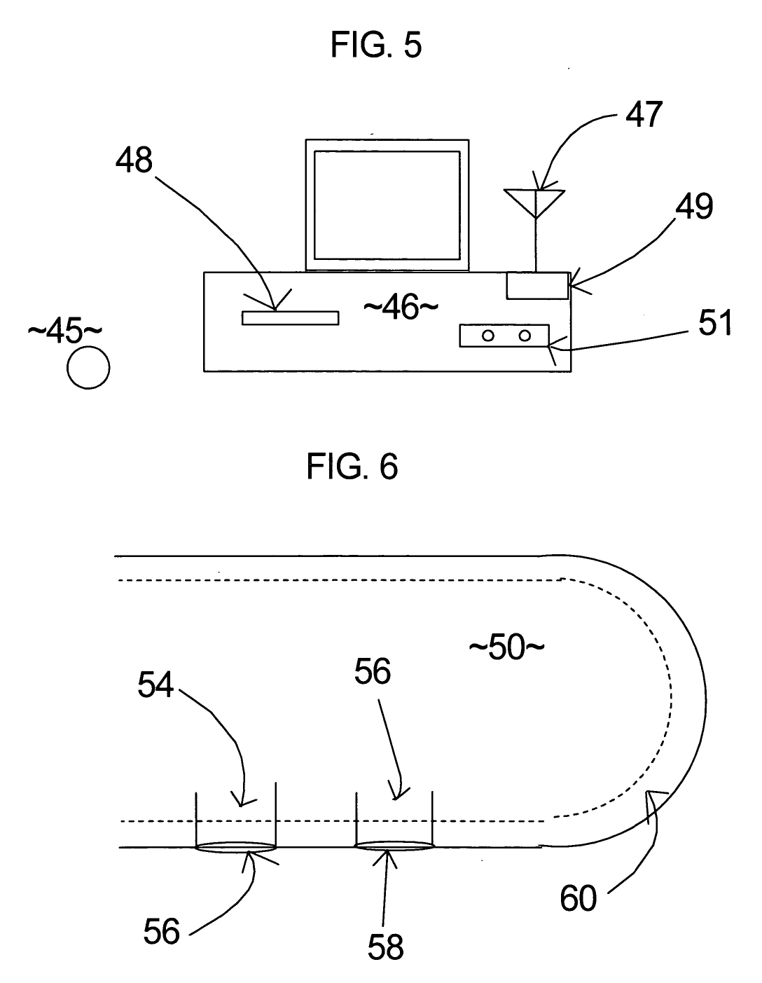

FIG. 2 is a schematic drawing presenting the components of the preferred embodiment of the present invention. The capsule 8 holds several components comprising the UWB imaging radar system. The battery 18 supplies power to the radio transceiver 16, controlling circuitry 14, and UWB radar sensor circuitry 12. The emitting and receiving antennae for the UWB radar circuitry are shown at 10 and 20 respectively. Alternatively, both UWB signal emitting and receiving could be done using one antenna only, depending on the imaging characteristics the circuit is designed to achieve. The controlling component 14 has memory and can be programmed by the computer system FIG. 5, described below, to perform desired actions.

The UWB radar sensor capsule imaging system shown in FIG. 2 is operated in the following manner:

When all the circuitry is assembled and power is supplied by the battery 18, the UWB imaging system is turned on and activated and sealed within the swallowable capsule shell 8. The programming in the controlling circuitry 14 uses the radio transceiver 16 to communicate with the computer system FIG. 5 to receive instructions regarding what actions to take.

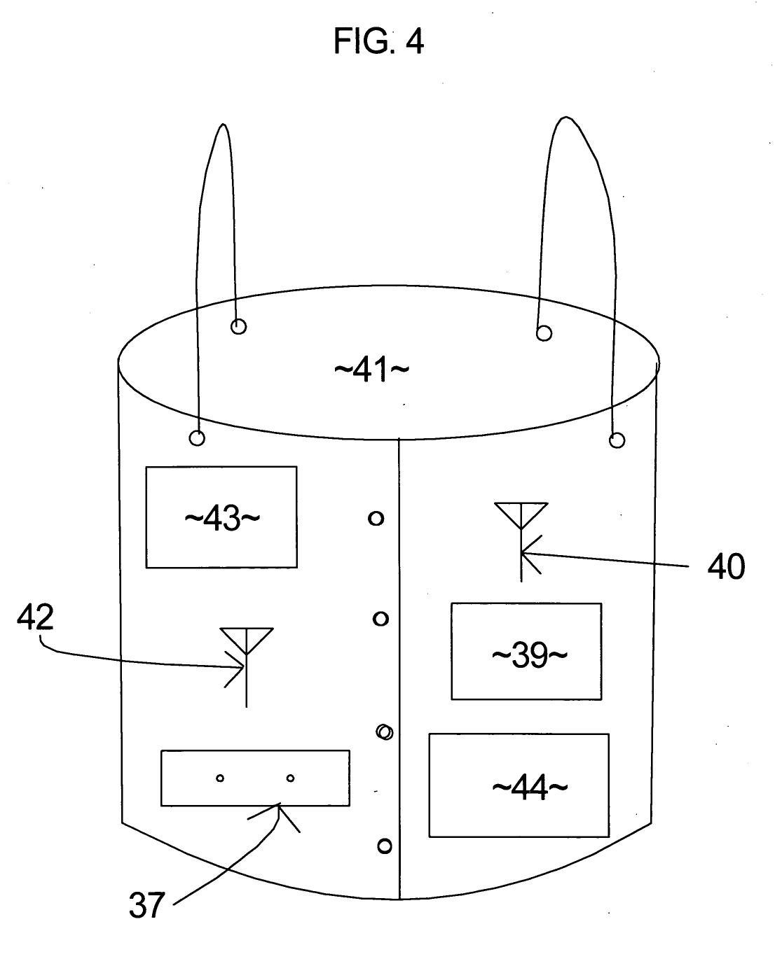

The computer system FIG. 5 uses its software programs and wireless communication to command the UWB radar capsule system to begin imaging by emitting and receiving UWB signals. The characteristics of the reflected UWB imaging signals are then transmitted from the capsule to the vest-style receiving device illustrated in FIG. 4 described below. The computer system FIG. 5 is also able to communicate with the controlling circuitry of the vest-style receiving device FIG. 4 to ensure its interoperability with the capsule device.

Typically the UWB capsule imaging system FIG. 2 is swallowed by the subject after it is turned on. The computer system FIG. 5 can further communicate with the capsule imaging system by means of the transceiver unit 16, to issue commands to it and monitor it as it travels through the GI tract.

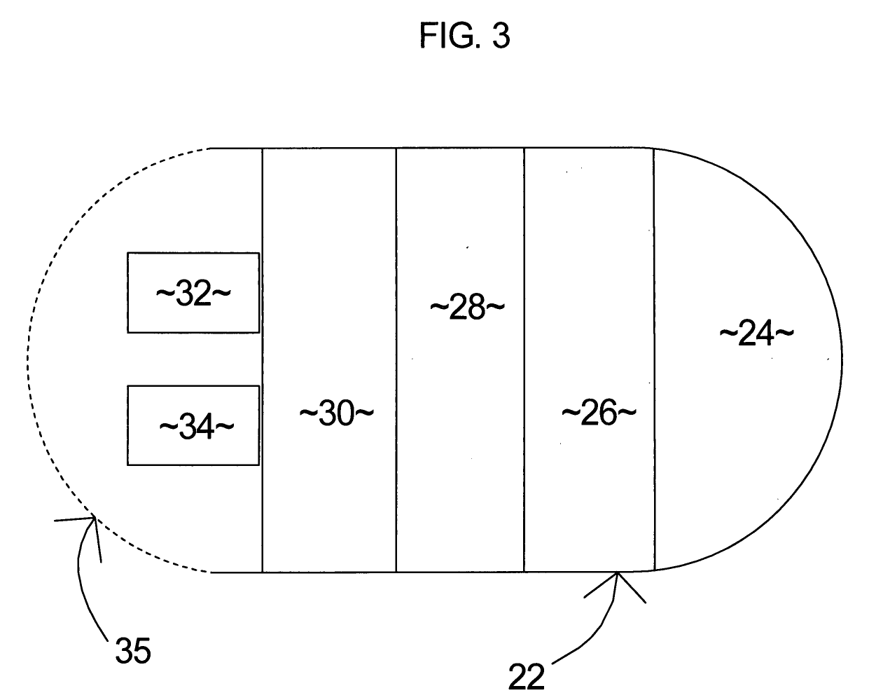

FIG. 3 is a schematic illustration of an alternative embodiment of the present invention. This alternative embodiment is comprised of components similar to the preferred embodiment—components such as the battery 24, transceiver unit 26, controlling unit 28, and the UWB radar sensor circuitry 30. However, in this alternative the UWB emitting and receiving antennae shown in FIG. 2 10 and 20, are not included. Instead an ultraviolet (UV) LED FIG. 3 32 is used to emit imaging waves, and an UV photodiode detector 34 is used to receive the reflected signals. Further, in order to allow the efficient passage of UV signals through the capsule shell, a cap that is made of UV wave transparent material 35 is used. The capsule 22 could be made entirely of material that is transparent to UV waves.

The operation of the alternative embodiment FIG. 3 is similar to that of the preferred embodiment described above. Here, however, the UWB circuitry is used to drive the UV LED 32 to emit UV signals rather than UWB signals within the regulated frequency range. Moreover, the reception of the UV signals reflected from the surrounding tissues is timed by the UWB circuitry driving the UV photodiode detector 34, similar to the manner that the UWB circuitry drives the timing of the receiving antenna FIG. 2 20 in the preferred embodiment, following common operation of UWB radar sensor systems.

Regarding FIG. 4, the wearable vest-style garment receiving system is shown. The vest 41 supports a plurality of embedded antennae like those at 40 and 42. It can also include a communication port 37 for direct wire connection to the computer system FIG. 5. A transceiver component 39 is included which is used for wireless communication. Moreover the vest garment supports a power supply 43 and a data recording device 44. All of these components are electrically connected to comprise the receiving system which collects signals broadcast by the capsule imaging device and stores this imaging data for processing by the computer system. The subject wears the vest-style receiving system as the capsule travels through the GI tract.

The receiving system FIG. 4 operates by receiving radio signals from the capsule transceiver FIG. 2 16, these radio signals relate the imaging information generated by the UWB radar sensor FIG. 2 12. When the radio signals from the capsule are gathered by the antennae embedded in the vest 40 and 42, they are relayed to the storage device 44 to save the information for processing by the computer system FIG. 5. The components comprising the receiving system are connected electrically by wiring not shown.

FIG. 5 is a representation of the computer system that is used to communicate with and command the capsule imaging device FIG. 2 and process the imaging data stored by the receiving system FIG. 4. Moreover, the computer system can communicate with the receiving system to ensure it is operating correctly. The computer system display and box 46 include an input port 48 that accepts a storage media object 45—such as a CD or magnetic disk—as input. This media input object 45 would be from the receiving system storage device FIG. 4 44 and it holds the radar imaging information. By inserting the storage media object into the computer at the reader input port 48, the imaging data is transferred to the computer system.

The computer system also includes an antenna 47 that is part of a transceiver 49 which is used to communicate wirelessly with the transceiver component of the capsule FIG. 2 16 and the transceiver component of the receiving station FIG. 4 39.

Further, the computer system FIG. 5 can include a communication port 51 that provides means for direct wire connection between the computer system and a capsule imaging system equipped with externally accessible electrodes as described in reference to FIG. 6. The computer can program the controlling circuitry of the capsule using this direct wire connection. Similarly, the computer could communicate with the receiving system communication port FIG. 4 37 by this means, rather than wirelessly or by using the data storage disk 45.

The computer system FIG. 5 runs software programs that process the imaging data into output formats like graphs, charts, tables, and pictures. This allows interested parties to view the imaging information in summary forms. For example, the computer could present a video image of the inside of the GI tract and close by tissues, as detected by the UWB sensor within the capsule as it travels the length of the GI tract.

FIG. 6 illustrates an alternative means that the computer system could use to communicate with the capsule imaging device. Here the capsule 50 is shown with two internal electrodes 54 and 56 passing through the shell of the capsule 60 to be exposed externally on the outside surface at 56 and 58 respectively. There can be a plurality of electrodes or a single one depending on design. These electrodes are electrically connected to the UWB imaging system circuitry contained inside the capsule. The computer system can be electrically connected to the capsule circuitry by wiring its communication port FIG. 5 51 to the electrodes 56 and 58. By this means the computer system could program and issue instructions to the controlling circuitry FIG. 2 14 within the capsule.

Although specific embodiments of the present invention have been described in detail above, it will be recognized by those skilled in the art that the present invention is not limited by these particular characteristics. In fact, variations can be made to the embodiments described without varying from the spirit of the present invention. Rather the scope of the present invention is defined only by the following claims:

I claim:

1. An imaging system comprising:
a swallowable capsule comprising:
an ultra-wideband radar sensor system for imaging objects; and
controlling circuitry means that operatively regulates said imaging system; and
a transceiver to transmit imaging signals of said ultra-wideband radar sensor system and to receive controlling signals; and
a power supply for said imaging system.

2. A system according to claim 1 and including a miniature communications port wherein the electrical circuitry within said capsule is connected through the capsule wall to electrical contacts located on the outside surface of the capsule wall.

3. A system according to claim 1, and alternative claim 2, and including a reception system, operatively connected to said imaging system, which receives said transmitted imaging signals comprising:
a plurality of antennae; and
a power supply; and
storage means; and
controlling circuitry means operatively connecting the parts of said reception system.

4. A system according to claim 3 and including a transceiver component for wireless communication with other systems.

5. A system according to claim 3 and including a communications port for direct wire communication with other systems.

6. A system according to claim 3 and including:
a communications port for direct wire communication with other systems; and
a transceiver component for wireless communication with other systems.

7. A system according to claim 3, and alternative claims 4 and 5 and 6, and including a programmable computer system operatively connected to said reception system and said imaging system, which processes said transmitted imaging signals saved in said storage means and controls said imaging system comprising:
a computer system; and
software programs which process said imaging signal data into various presentation formats; and
software programs to issue instructions to said controlling circuitry means of said imaging system; and
input means; and
transmission means operatively connecting said computer system input means with said storage means of said reception system.

8. A system according to claim 7 and including a transceiver component for wireless communications with said transceiver component of said capsule and said transceiver component of said reception system.

9. A system according to claim 7 and including a communications port for direct wire connections with said miniature communications port of said capsule and said communications port of said reception system.

10. A system according to claim 7 and including:
a transceiver component for wireless communications with other systems; and
a communications port for direct wire connections to other systems.

11. A system according to claim 1 and alternative claim 2, wherein the electromagnetic wave emitter of said ultra-wideband radar sensor system is an ultraviolet frequency light emitting diode and the electromagnetic wave receiver is an ultraviolet frequency sensitive detector and the shell of said capsule is transparent to ultraviolet waves.

12. A system according to claim 1 and alternative claim 2, wherein the electromagnetic wave emitter of said ultra-wideband radar sensor system is an infrared frequency light emitting diode and the electromagnetic wave receiver is an infrared frequency sensitive detector and the shell of said capsule is transparent to infrared waves.
